(19) United States
(12) Patent Application Publication
(10) Pub. No.: US 2025/0017169 A1
(43) Pub. Date: Jan. 16, 2025

(54) REPTILE PET BREEDING CABINET ASSEMBLY AND REPTILE PET BREEDING CABINET

(71) Applicant: DA VINCI PET INTELLIGENCE (SHENZHEN) CO., LTD., SHENZHEN CITY (CN)

(72) Inventor: ZETAO GUO, JINZHONG CITY (CN)

(21) Appl. No.: 18/625,153

(22) Filed: Apr. 2, 2024

(30) Foreign Application Priority Data
Jul. 14, 2023 (CN) 202321846941.8
Sep. 27, 2023 (CN) 202322627280.6

(51) Int. Cl. A01K 1/03; A01K 67/00

(52) U.S. Cl. CPC A01K 1/03; A01K 67/00

(57) ABSTRACT

A reptile pet breeding cabinet assembly and a reptile pet breeding cabinet related to the technical field of reptile equipment are provided. The reptile pet breeding cabinet assembly includes a breeding box, a layer plate and a heating element. The breeding box is configured for breeding reptile pets, the layer plate is provided on a lower side of the breeding box, and the layer plate is provided with a heat preservation area for containing gas. The heating element is provided between the breeding box and the layer plate, and configured to heat a bottom of the breeding box and cover the heat preservation area.

### FIELD OF THE DISCLOSURE

The present disclosure relates to the technical field of reptile equipment, and more particularly to a reptile pet breeding cabinet assembly and a reptile pet breeding cabinet.

### BACKGROUND OF THE DISCLOSURE

Reptile pets refer to a new fashion of feeding reptiles as pets, and Arthropoda (such as Crustacea) and Amphibians, which are similar to reptiles, also joined the ranks of reptile pets. The characteristics of reptile pets are that reptile pets are all poikilotherm animals, live on land (some are amphibious) and rely on crawling for activities.

Reptile pet breeding cabinets are usually provided with external heating devices, and electric hot plates are usually applied to the existing heating devices used in reptile cabinets, but a lot of heat is dissipated. Although the heat loss can be reduced by adding insulation cottons, the cost is high, so that it is urgently needed to improve.

### SUMMARY OF THE DISCLOSURE

The purpose of the present disclosure is to provide a reptile pet breeding cabinet assembly and a reptile pet breeding cabinet in view of the defects and shortcomings of the existing technology, which have the advantages of reducing cost and reducing heat loss.

In order to achieve the above objects, the technical solution adopted by the present disclosure is as follows. A reptile pet breeding cabinet assembly is provided, which includes a breeding box configured to breed reptiles; a layer plate provided on a lower side of the breeding box, in which a heat preservation area for containing gas is provided on the layer plate; and a heating element provided between the breeding box and the layer plate, and configured to heat a bottom of the breeding box and to cover the heat preservation area.

Further, the layer plate includes a bottom layer plate and a top layer plate. The bottom layer plate is provided on the lower side of the breeding box, the top layer plate is provided on an upper side of the breeding box and is configured to suspend the breeding box. The top layer plate is configured to be the bottom layer of another breeding box arranged above the breeding box.

Further, a convex structure is provided on the layer plate, the heat preservation area is formed in the convex structure, the layer plate is provided with a ventilation area on a side of the heat preservation area, and a top side of the convex structure is higher than the ventilation area.

Further, the ventilation area includes a front ventilation area located on a front side of the heat preservation area, and a rear ventilation area located on a rear side of the heat preservation area.

Further, the front ventilation area is configured to be adjacent to a pedestrian passage, and the heating element is a flexible heating sheet located on a side away from the pedestrian passage.

Further, a side of the ventilation area connected to the convex structure is in an arc-shaped transition.

Further, a sliding slot is provided one each of a left side and a right side of a lower part of the layer plate, an outer edge slidably assembled on the sliding slot is provided on each of a left side and a right side of an upper part of the breeding box, and the breeding box is configured to be suspended on the top layer plate and capable of being pulled outward relative to the top layer plate.

Further, the reptile pet breeding cabinet assembly includes a box cover covering the upper side of the breeding box and located on a lower side of the top layer plate, and a plurality of ventilation holes are provided on the box cover.

Further, an overlapping rib that protrudes inward is provided on an inner wall of the breeding box adjacent to the upper side of the breeding box, and the box cover is configured to be placed on the overlapping rib.

Further, a limiting notch is provided on the overlapping rib, and a limiting protrusion corresponding to the limiting notch is provided on a bottom side of the box cover.

Further, the reptile pet breeding cabinet assembly includes a partition configured to divide the breeding box into at least two spaces.

In order to achieve aforementioned purpose, another technical solution adapted by the present disclosure is to provide a reptile pet breeding cabinet, which includes a frame; a plurality of layer plates provided at different heights of the frame to form a multi-layer space, in which heat preservation areas for containing gas are respectively provided on the plurality of layer plates; a plurality of breeding boxes correspondingly suspended on the plurality of layer plates, in which each of the plurality of breeding boxes is located between an upper one of the layer plate and a lower one of the layer plate and configured to breed reptiles; a plurality of heating elements correspondingly provided between the plurality of breeding boxes and the plurality of layer plates, and configured to heat bottoms of the plurality of breeding boxes respectively arranged above the plurality of heating elements and configured to cover the heat preservation areas; and a temperature adjustment device correspondingly and wirelessly or wiredly connected to the plurality of heating elements, and configured to adjust temperatures of the plurality of heating elements.

Further, multiple layer plates are provided side by side at a same height, one of the plurality of heating elements is provided on one of the multiple layer plates on a same layer, and each of the multiple heating elements corresponds to a corresponding one of the plurality of breeding boxes.

Further, the frame includes a plurality of left front connecting members each having inserting blocks respectively facing a right side, a rear side, an upper side and a lower side of the reptile pet breeding cabinet; a plurality of right front connecting members each having inserting blocks respectively facing the left side, the rear side, the upper side and the lower side of the reptile pet breeding cabinet; a plurality of left rear connecting members each having inserting blocks respectively facing the right side, the front side, the upper side and the lower side of the reptile pet breeding cabinet; a plurality of right rear connecting members each having inserting blocks respectively facing the left side, the front side, the upper side and the lower side of the reptile pet breeding cabinet; a plurality of front horizontal connecting rods each having one end sleeved outside the left front connecting member, and another end sleeved on the right front connecting member on a same layer; a plurality of rear horizontal connecting rods each having one end sleeved outside the left rear connecting member, and another end sleeved on the right rear connecting member on a same layer; a plurality of left horizontal connecting rods each having one end sleeved outside the left front connecting member, and another end sleeved on the left rear connecting member on a same layer; a plurality of right horizontal connecting rods each having one end sleeved outside the right front connecting member, and another end sleeved on the right rear connecting member on a same layer; a plurality of left front vertical connecting rods each having one end sleeved outside one of the plurality of left front connecting members, and another end sleeved outside another of the plurality of left front connecting members that is adjacent to the left front vertical connecting rod; a plurality of right front vertical connecting rods each having one end sleeved outside one of the plurality of right front connecting members, and another end sleeved outside another of the plurality of right front connecting members that is adjacent to the right front vertical connecting rod; a plurality of left rear vertical connecting rods each having one end sleeved outside one of the plurality of left rear connecting members, and another end sleeved outside another of the plurality of left rear connecting members that is adjacent to the left rear vertical connecting rod; and a plurality of right rear vertical connecting rods each having one end sleeved outside one of the plurality of right rear connecting members, and another end sleeved outside another of the plurality of right rear connecting members that is adjacent to the right rear vertical connecting rod.

Further, each of the plurality of left front connecting members includes a base body in the shape of a cube; an extension plate extending forward from a left end surface of the base body, in which the extension plate is aligned with an end of one of the plurality of front horizontal connecting rods; a right inserting block protruding from a right end surface toward a right side of the extension plate and configured for the front horizontal connecting rod to be inserted into; a rear inserting block protruding rearward from a rear end surface of the base body and configured for the left horizontal connecting rod to be inserted into; an upper inserting block protruding upward from an upper end surface of the base body and configured for the left front vertical connecting rod to be inserted into; and a lower inserting block protruding downward from a lower end surface of the base body and configured for the left front vertical connecting rod to be inserted into. The plurality of front horizontal connecting rods, the plurality of rear horizontal connecting rods, the plurality of left horizontal connecting rods, the plurality of right horizontal connecting rods, the plurality of left front vertical connecting rods, the plurality of right front vertical connecting rods, the plurality of left rear vertical connecting rods and the plurality of right rear vertical connecting rods are all cuboid-shaped and have a square cross-section.

Further, the plurality of front horizontal connecting rods, the plurality of rear horizontal connecting rods, the plurality of left horizontal connecting rods, the plurality of right horizontal connecting rods, the plurality of left front vertical connecting rods, the plurality of right front vertical connecting rods, the plurality of left rear vertical connecting rods and the plurality of right rear vertical connecting rods all includes a rod body, in which a plurality of through-slots formed in a circle along a length direction and configured for inserting are provided on the rod body, and one of the plurality of through-slots is not closed in the length direction, and a locking hole is provided between the through-slots.

Further, two right inserting blocks are provided, and a first through hole between the two right inserting blocks is provided on the extension plate. When the plurality of front horizontal connecting rods are inserted into the left front connecting member, a locking member is used to pass through the first through hole and the locking hole of the front horizontal connecting rod for locking. Two rear inserting blocks are provided, a second through hole between the two rear inserting blocks is provided on the base body. When the left horizontal connecting rod is inserted into the left front connecting member, the locking member is used to pass through the second through hole and the locking hole of the left horizontal connecting rod for locking. Three upper inserting blocks are provided in a circle, and a third through hole is provided on one of the upper inserting blocks corresponding to the through-slot that is not closed in the length direction in a horizontal direction. When a lower end of the left front vertical connecting rod is inserted into the left front connecting member, the locking member is used to pass through the third through hole for locking. Three lower inserting blocks are provided in a circle, and a fourth through hole is provided on one of the lower inserting block corresponding to the through-slot that is not closed in the length direction in the horizontal direction. When an upper end of the left front vertical connecting rod is inserted into the left front connecting member, the locking member is used to pass through the fourth through hole for locking.

Further, a sliding bar is provided on each of a front side and a rear side of the layer plate corresponding to the through-slot that is not closed in the length direction. When the layer plate is slidably assembled between the front horizontal connecting rod and the rear horizontal connecting rod, the layer plate is suspended on the frame body by the front horizontal connecting rod and the rear horizontal connecting rod.

Further, a temperature sensor is provided on each of the plurality of heating elements and/or the breeding box, a display screen is provided on the temperature adjustment device, the temperature adjustment device is configured to adjust a temperature and display the temperature on the display screen, and the temperature adjustment device and the plurality of heating elements are independently controlled.

Further, a light sensor and/or a time module signally connected to the temperature adjustment device is provided on the reptile pet breeding cabinet, the temperature adjustment device adjusts temperatures of the plurality of heating elements through signals fed back by the light sensor and/or the time module, so that temperatures in the plurality of breeding boxes are adjusted.

By virtue of the above technical solution, the beneficial effects of the present disclosure are as follows.

1. In the present disclosure, the layer plate is provided at the bottom of the breeding box, the layer plate has the heat preservation area for containing the gas, and the heating element covering the heat preservation area is provided between the breeding box and the layer plate, so that when used, part of heat generated by the heating element is transferred to the layer plate, and the heat preservation area sealed with air in the layer plate can insulate the heat and then feed the heat back to the heating element and the breeding box, which greatly reduces the heat loss of the heating element, and the air is a free insulation material, so that the cost is reduced.

2. In the present disclosure, in addition to being used for placing heating elements and having a heat insulation effect, the layer plate can also serve as the top layer plate of the next layer of breeding box, so that it can be used for suspending the next layer of breeding box, thereby eliminating the need to provided two sets of molds to produce bottom layer plates and top layer plates respectively.

3. In the present disclosure, the frame body includes the plurality of left front connecting members, the plurality of right front connecting members, the plurality of left rear connecting members, the plurality of right rear connecting members, the plurality of front horizontal connecting rods, the plurality of rear horizontal connecting rods, the plurality of left horizontal connecting rods, the plurality of right horizontal connecting rods, the plurality of front vertical connecting rods, the plurality of right front vertical connecting rods, the plurality of left rear vertical connecting rods and the plurality of right rear vertical connecting rods. These components are disassembled for easy packaging and transportation, and when a user wants to use them, the connecting rods can be inserted into the corresponding connecting members respectively. After that, the frame can be formed, and assembly is also more convenient.

### BRIEF DESCRIPTION OF THE DRAWINGS

In order to explain the embodiments of the present disclosure or the technical solutions in the prior art more clearly, the drawings needed to be used in the description of the embodiments or the prior art will be briefly introduced below. Obviously, the drawings in the following description are only some embodiments of the present disclosure. For those of ordinary skill in the art, other drawings can be obtained based on these drawings without exerting any creative effort.

FIG. 1 is a schematic structural diagram of a reptile pet breeding cabinet assembly;

FIG. 2 is a schematic structural diagram of the reptile pet breeding cabinet assembly with a top layer plate;

FIG. 3 is a schematic exploded diagram of the structure in FIG. 2;

FIG. 4 is a schematic structural diagram of the layer plate;

FIG. 5 is a schematic structural diagram of a sliding assembly of a breeding box and the layer plate;

FIG. 6 is a schematic structural diagram of the breeding box and a box cover;

FIG. 7 is an enlarged view of part A in FIG. 6;

FIG. 8 is an enlarged view of part B in FIG. 6;

FIG. 9 is a schematic structural diagram of the breeding box and a partition;

FIG. 10 is an enlarged view of part C in FIG. 9;

FIG. 11 is a schematic structural diagram of the reptile pet breeding cabinet;

FIG. 12 is a schematic structural diagram of the reptile pet breeding cabinet from another perspective;

FIG. 13 is an enlarged view of part D in FIG. 12;

FIG. 14 is a schematic structural diagram of a left front connecting member, a front horizontal connecting rod, a left horizontal connecting rod, and two right front vertical connecting rods;

FIG. 15 is an enlarged view of part E in FIG. 14;

FIG. 16 is a schematic structural diagram of the left front connecting member, the front horizontal connecting rod, the left horizontal connecting rod, and the two right front vertical connecting rods from another perspective;

FIG. 17 is an enlarged view of part F in FIG. 16;

FIG. 18 is a structural schematic diagram of the left front connecting member, the front horizontal connecting rod, the left horizontal connecting rod, and the two right front vertical connecting rods from yet another perspective;

FIG. 19 is an enlarged view of part G in FIG. 18;

FIG. 20 is a schematic structural diagram of the left rear connecting member;

FIG. 21 is a schematic structural diagram of the left lower front connecting member;

FIG. 22 is a schematic structural diagram of the layer plate, the front horizontal connecting rod, and the rear horizontal connecting rod;

FIG. 23 is a cross-sectional view taken along A-A in FIG. 22;

FIG. 24 is an enlarged view of part H in FIG. 23;

FIG. 25 is a schematic diagram of a parallel circuit of a heating system of the reptile pet breeding cabinet; and

FIG. 26 is a schematic diagram of joint connections of the heating system of the reptile pet breeding cabinet.

Reference numeral: 100, breeding box; 110, outer edge; 120, front baffle; 130, overlapping rib; 131, limiting notch; 140, limiting clip; 200, layer plate; 200a, bottom layer plate; 200b, top layer plate; 210, convex structure; 211, heat preservation area; 220, ventilation area; 220a, front ventilation area; 220b, rear ventilation area; 230, sliding slot; 240, sliding bar; 300, heating element; 310, heating wire; 320, temperature sensor; 400, box cover; 410, limiting protrusion; 500, partition; 600, frame body; 610, connecting member; 610a, left front connecting member; 611a, base body; 6111a, second through hole; 612a, extension plate; 6121a, first through hole; 613a, right inserting block; 614a, rear inserting block; 615a, upper inserting block; 6151a, third through hole; 616a, lower inserting block; 6161a, fourth through hole; 610b, right front connecting member; 610c, left rear connecting member; 611c, base body; 613c, right inserting block; 614c, front inserting block; 615c, upper inserting block; 616c, lower inserting block; 610d, right rear connecting member; 610e, lower left front connecting member; 611e, base body; 612e, extension plate; 613e, wheel connecting plate; 620, connecting rod; 620a, front horizontal connecting rod; 621a, rod body; 622a, through-slot; 623a, locking hole; 620b, rear horizontal connecting rod; 620c, left horizontal connecting rod; 621c, rod body; 622c, through-slot; 623c, locking hole; 620d, right horizontal connecting rod; 620e, left front vertical connecting rod; 621e, rod body; 622e, through-slot; 623e, locking hole; 620f, right front vertical connecting rod; 620g, left rear vertical connecting rod; 620h, right rear vertical connecting rod; 700, temperature adjustment device; 800, wheel; 900, pressing plate.

### DETAILED DESCRIPTION OF THE EXEMPLARY EMBODIMENTS

The present disclosure will be further described in detail below with reference to the accompanying drawings.

This specific embodiment is only an explanation of the present disclosure, and is not a limitation of the present disclosure. Those skilled in the art can make modifications to this embodiment without creative contribution as needed after reading this description, and as long as they are within the scope of the claims of the present disclosure, they are protected by the patent law.

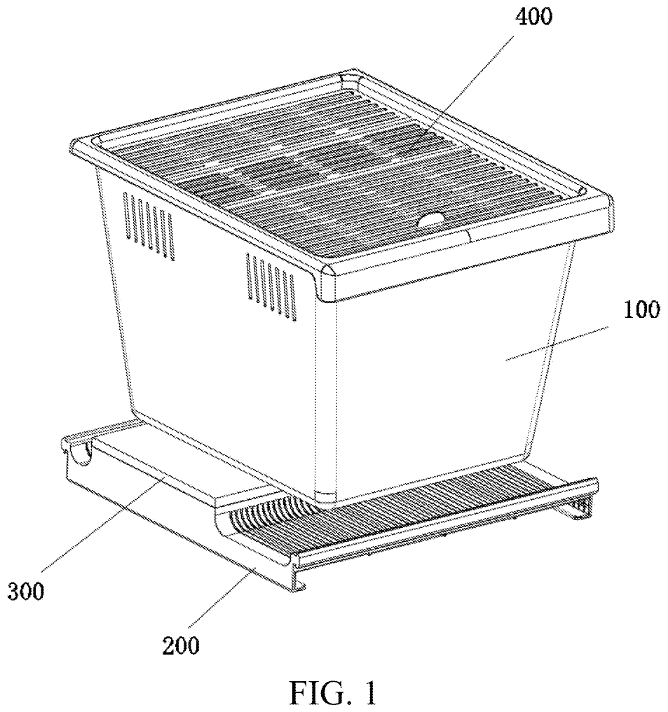

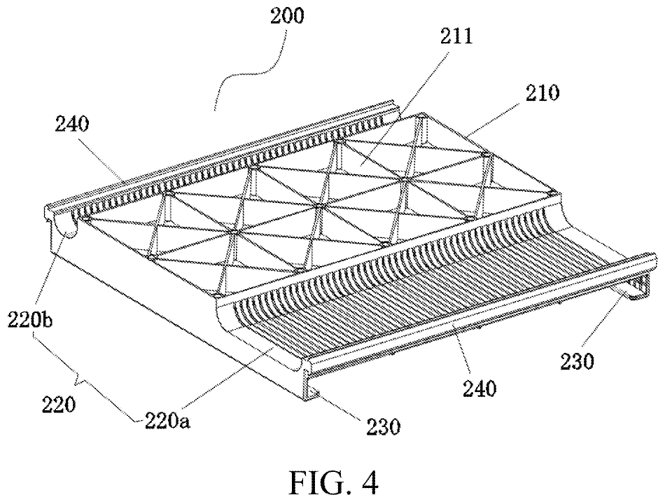

The present embodiment relates to a reptile pet breeding cabinet assembly. Referring to FIGS. 1 and 4, the reptile pet breeding cabinet assembly includes a breeding box 100, a layer plate 200 and a heating element 300. The breeding box 100 is used for breeding reptiles, and a plurality of ventilation holes are provided on a side wall of the breeding box 100. The layer plate 200 is provided on a lower side of the breeding box 100, and the layer plate 200 is provided with a heat preservation area 211 for containing gas. The heating element 300 is provided between the breeding box 100 and the layer plate 200, and used for heating a bottom of the breeding box 100 and covering the heat preservation area 211. The heating element 300 can provide a stable temperature for the breeding box 100 to facilitate raising and breeding of reptiles. The layer plate 200 is provided at the bottom of the breeding box 100 and provided with the heat preservation area 211 for containing the gas, and the heating element 300 covering the heat preservation area 211 is provided between the breeding box 100 and the layer plate 200, so that when used, part of heat generated by the heating element 300 is transferred to the layer plate 200, and the heat preservation area 211 sealed with air in the layer plate 200 can insulate the heat, and then feed the heat back to the heating element 300 and the breeding box 100, which greatly reduces the heat loss of the heating element 300, and the air is a free heat insulation material, so that the cost is reduced.

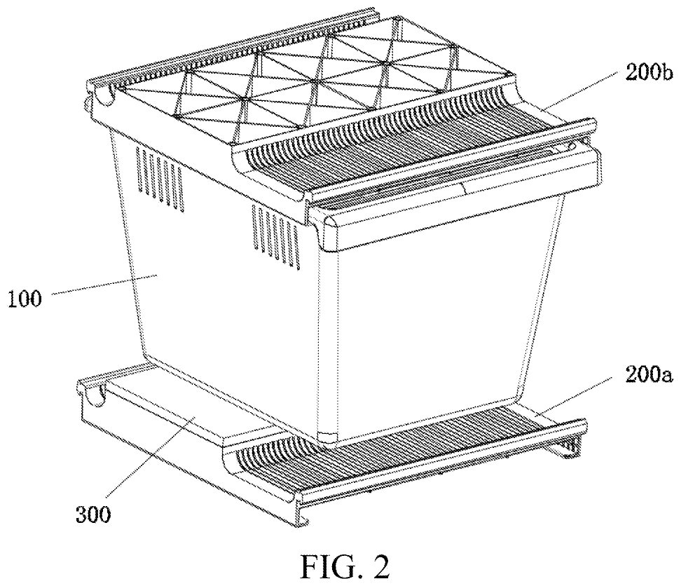

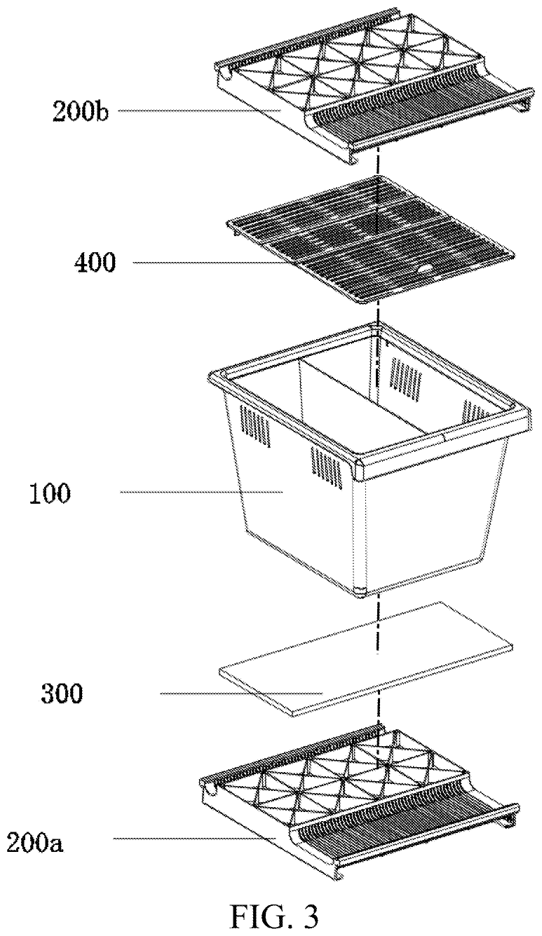

On this basis, referring to FIGS. 2 and 3, the layer plate 200 includes a bottom layer plate 200a and a top layer plate 200b. The bottom layer 200a is provided on the lower side of the breeding box 100, the top layer plate 200b is provided on an upper side of the breeding box 100 and used for suspending the breeding box 100, and the top layer plate 200b is the bottom layer 200a of an upper layer of the breeding box 100. That is to say, the bottom layer 200a, in addition to being used for placing the heating element 300 and maintaining the heat, can also serve as the top layer plate 200b of the next layer of the breeding box 100, which can be used for suspending the next layer of the breeding box 100, thereby eliminating the need for two sets of molds to produce the bottom layer 200a and the top layer plate 200b respectively, further reducing the cost of mold opening.

Referring to FIG. 4, one layer plate 200 is taken as an example. The layer plate 200 has a convex structure 210, and the heat preservation area 211 with an open upper end is formed in the convex structure 210. The layer plate 200 is provided with a ventilation area 220 on a side of the heat preservation area 211, and a top side of the convex structure 210 is higher than the ventilation area 220. One of the purposes of raising the height of the convex structure 210 is to increase a volume of the heat preservation area 211, so that more free insulation material, i.e., the air, can be accommodated in the heat preservation area 211. The other purpose of raising the height of the convex structure 210 is to provide a larger gap between the ventilation area 220 and the breeding box 100 on an upper side of the ventilation area 220, so that it is more conducive to exchange of the gas between the breeding box 100 on a lower side of the ventilation area 220 and the outside air.

Referring to FIG. 4, a plurality of grids are provided in the heat preservation area 211, and an X-shaped reinforcing rib is provided in each of the plurality of grids, thereby greatly enhancing a structural strength of the layer plate 200.

Reference is further made to FIG. 4. Specifically, the ventilation area 220 includes a front ventilation area 220a located on a front side of the heat preservation area 211, and a rear ventilation area 220b located on a rear side of the heat preservation area 211. By providing the ventilation areas 220 one behind the other, it is more conducive to the circulation of the air in the breeding box 100 and the outside.

The reptile pet breeding cabinet assembly is a part of a reptile pet breeding cabinet. The reptile pet breeding cabinet assembly is installed behind a frame body 600 of the reptile pet breeding cabinet. The front ventilation area 220a is configured to be adjacent to a pedestrian passage, and the rear ventilation area 220b is configured to be adjacent to a wall. The heating element 300 does not completely cover a lower end surface of the breeding box 100, and only covers a part of the breeding box 100 and is closer to the wall, and the heating element 300 is closer to the wall, so that wires connected to the heating element 300 can be better hidden adjacent to the wall, and it is also convenient to connect to a socket on the wall without affecting the pedestrian passage. In addition, the heating element 300 is placed in such a position that there are different temperature zones in the breeding box 100 (a position adjacent to the heating element 300 has a high temperature, and a position far away from the heating element 300 has a low temperature), so that the reptile pet can choose activities in a more comfortable temperature zone according to its habits and preferences.

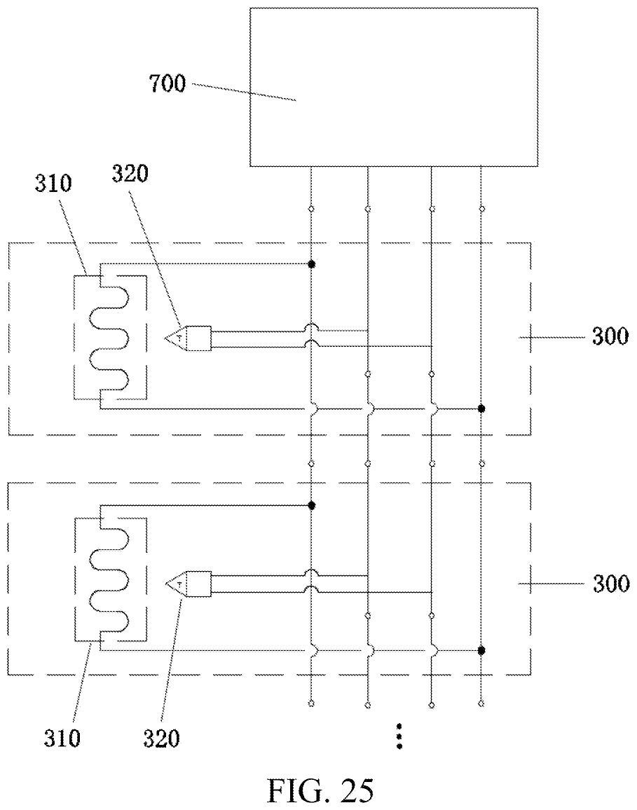

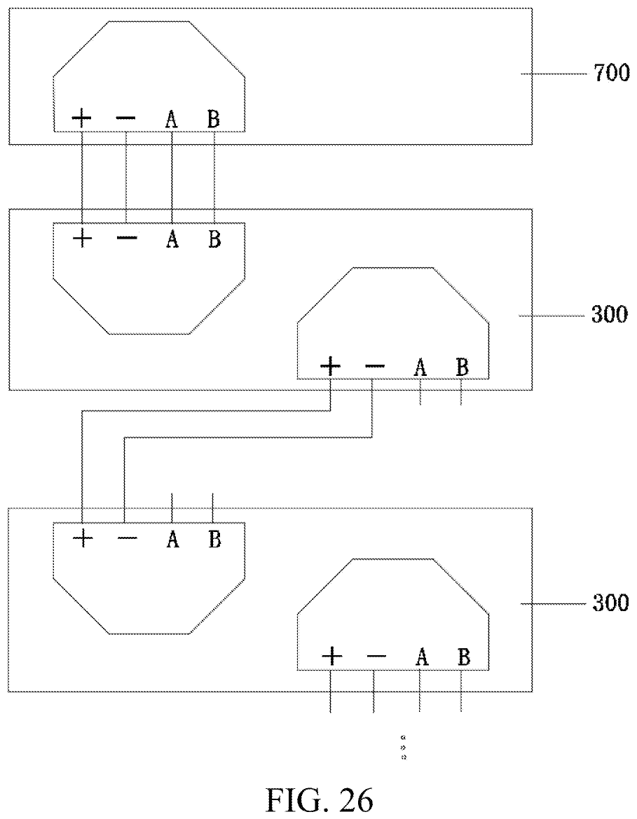

In the present embodiment, as shown in FIG. 25, the heating element 300 is a flexible heating sheet and specifically is leather used as the outer layer, and a plurality of heating wires 310 are provided in the heating element 300.

In the present embodiment, a plurality of ventilation holes are provided in both the front ventilation area 220a and the rear ventilation area 220b, and an area of the front ventilation area 220a is larger than an area of the rear ventilation area 220b. In other embodiments, the area of the rear ventilation area 220b can also be set to be larger than the area of the front ventilation area 220a.

The breeding box 100 in the present embodiment is larger at a top and smaller at a bottom, that is, an upper end surface of the breeding box 100 is larger than a lower end surface of the breeding box 100, and the heating element 300 is provided on one side closer to the wall, that is, the heating element 300 is located at a rear end of the bottom of the breeding box 100, so that the air temperature near the rear ventilation area 220b in the box 100 is higher than the air near the front ventilation area 220a. Since the cold air is heavier and the hot air is lighter, the hot air rises through the rear ventilation area 220b and is discharged through the rear ventilation area 220b, and the cold air enters the breeding box 100 through the front ventilation area 220a, thereby improving air circulation efficiency.

Reference is further made to FIGS. 4, 22 and 23. In the layer plate 200, one side of the ventilation area 220 connected to the convex structure 210 is in an arc-shaped transition. The convex structure 210 is higher than the ventilation area 220. The arc-shaped transition between the convex structure 210 and the ventilation area 220 makes the layer plate 200 a stronger structural strength than the direct right-angle transition. Further, the arc-shaped transition is more convenient for cleaning the dust scattered on the ventilation area 220. In addition, a side of the ventilation area 220 away from the convex structure 210 also adopts an arc-shaped protrusion, so that both the front ventilation area 220a and the rear ventilation area 220b have a U-shaped shape.

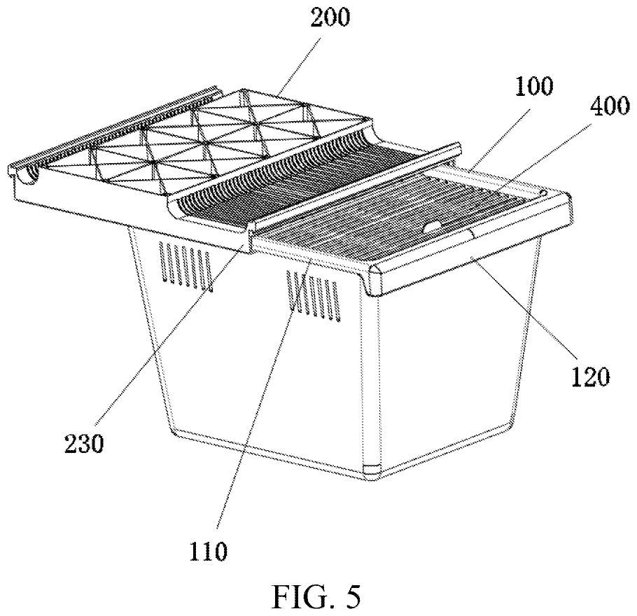

Referring to FIGS. 4 and 5, a sliding slot 230 is provided on each of a left side and a right side of a lower part of the layer plate 200, and is slidably assembled on an outer edge 110 of the sliding slot 230. By virtue of a cooperation between the outer edge 110 and the sliding slot 230, the breeding box 100 is suspended on the top layer plate 200b and can be pulled outward relative to the top layer plate 200b. A front baffle 120 that downwardly extends is provided on a front side of the outer edge 110 of the breeding box 100. The front baffle 120 prevents the breeding box 100 from sliding out to a rear side, and also facilitates a user to pull out the breeding box 100 forward.

Referring to FIGS. 3, 5, and 6, the reptile pet breeding cabinet assembly further includes a box cover 400 that covers the upper side of the breeding box 100 and is located under the top layer plate 200b. The box cover 400 is provided with a plurality of cover ventilation holes. Certain reptiles will cling to the side wall of the breeding box 100, or climb on stone or branches in the breeding box 100. If the box cover 400 is not provided, the tail of the reptile may be exposed on the top layer plate 200b or the tail may be caught between the top layer plate 200b and the breeding box 100. At this time, pulling the breeding box 100 may injure or frighten the reptile. This situation can be avoided by providing a box cover 400.

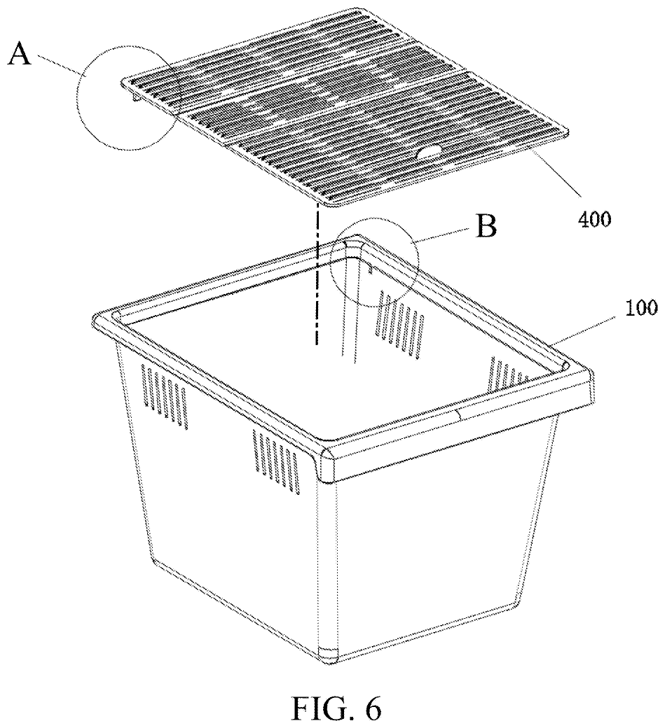

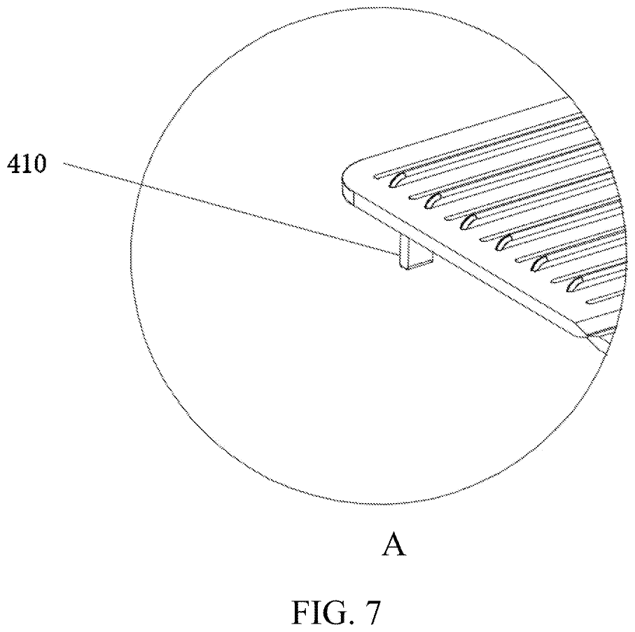

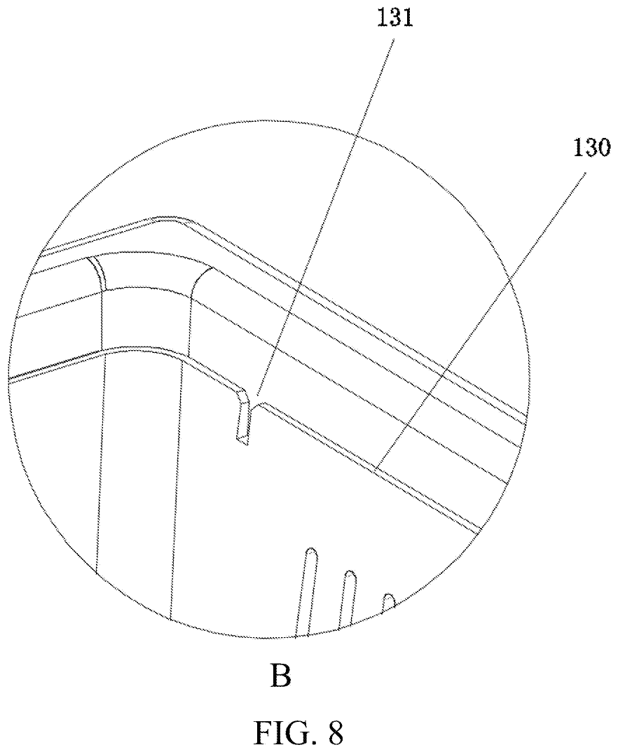

In order to further avoid that pulling the breeding box 100 may injure or frighten the reptile, the box cover 400 needs to be kept at a distance from the top layer plate 200b. Specifically, in the present embodiment, with reference to FIGS. 6 and 8, a part of the inner wall of the breeding box 100 adjacent to the upper side is provided with an overlapping rib 130 that inwardly protrudes, and the box cover 400 is placed on the overlapping rib 130. In order to make the box cover 400 more stable after being placed, with reference to FIGS. 6 to 8, the overlapping rib 130 is provided with a limiting notch 131, and the bottom side of the box cover 400 is provided with a limiting protrusion 410 corresponding to the limiting notch 131. When the box cover 400 is placed on the overlapping rib 130, the limiting protrusion 410 on the box cover 400 is embedded in the limiting notch 131 of the breeding box 100.

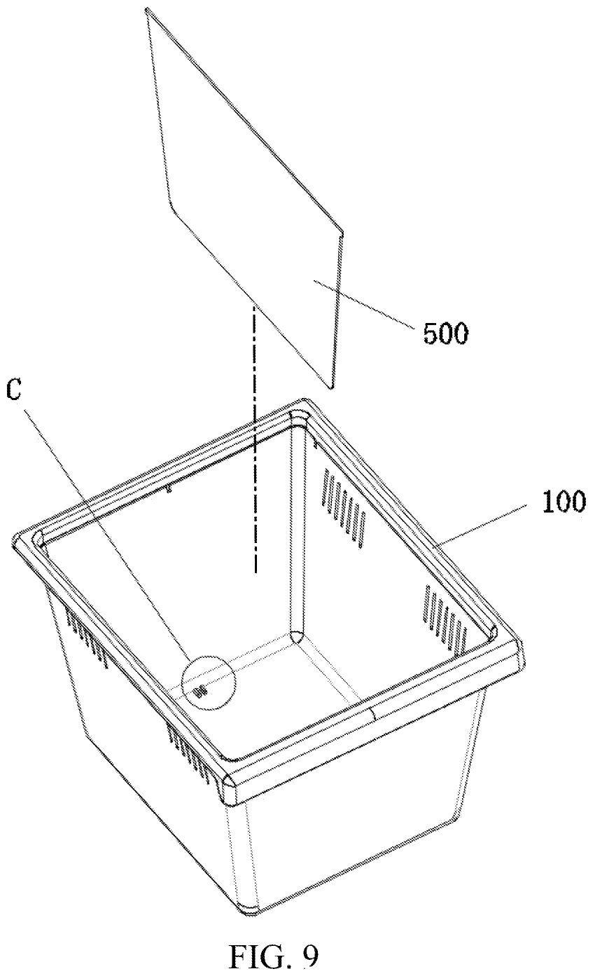

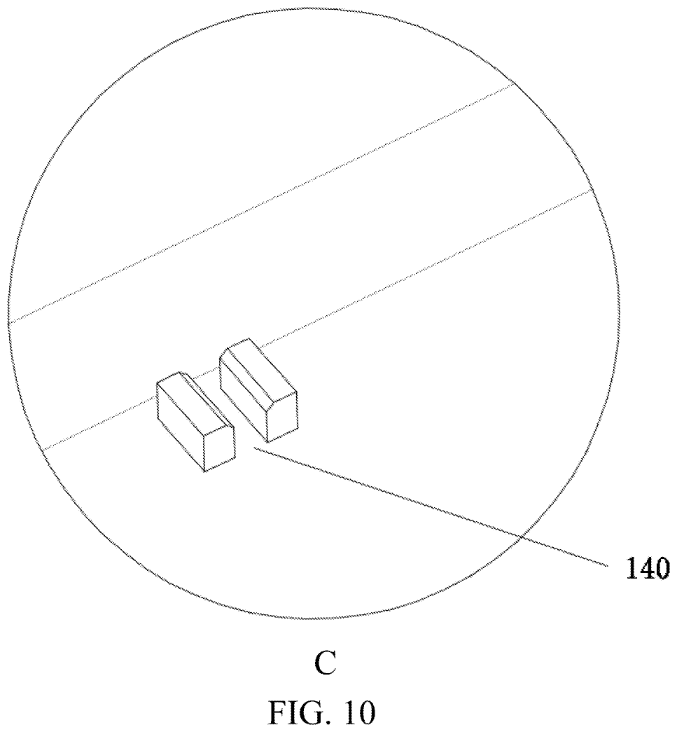

Referring to FIGS. 3 and 9, the reptile pet breeding cabinet assembly further includes a partition 500 for dividing the breeding box 100 into at least two spaces. The partition 500 in the present embodiment is in the shape of a flat plate, thereby dividing the breeding box 100 into two spaces. The partition 500 can also be set in a Y shape, so that the breeding box 100 can be divided into three spaces. The partition 500 can also be set in a cross shape, so that the breeding box 100 can be divided into four spaces. Further, referring to FIGS. 9 and 10, a limiting clip 140 is provided at the bottom of the breeding box 100, and the partition 500 is detachably inserted into the limiting clip 140. In certain embodiments, the limiting clip 140 can also be provided on the inner side wall of the breeding box 100. In addition, multiple limiting clips 140 can also be provided, so that the sizes of different separated spaces can be selected.

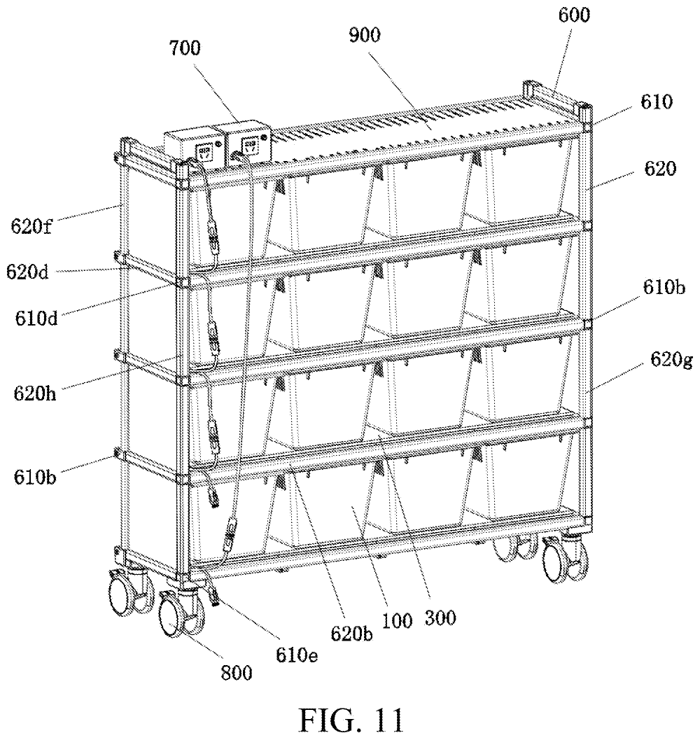

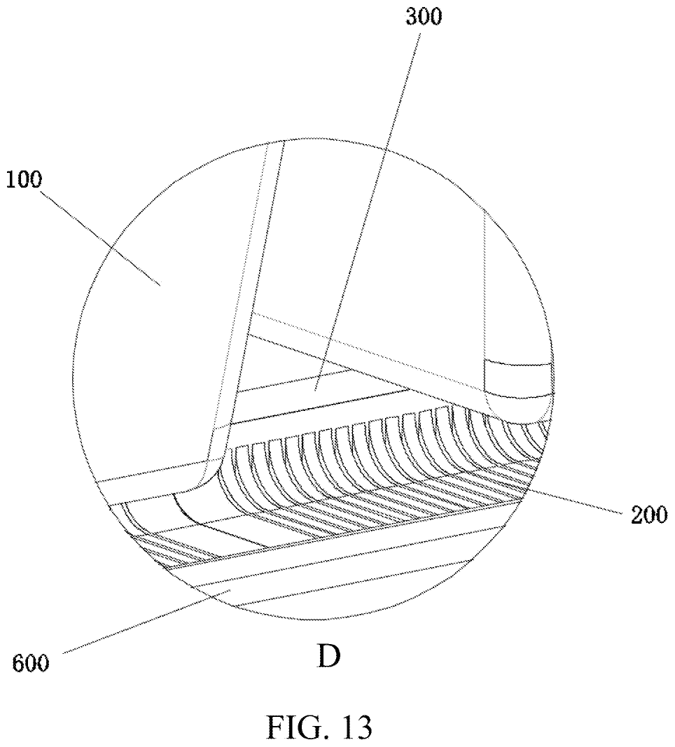

The present embodiment further relates to a reptile pet breeding cabinet. Referring to FIGS. 11 to 13, the reptile pet breeding cabinet includes a frame body 600, a plurality of layer plates 200, a plurality of breeding boxes 100, a plurality of heating elements 300 and a temperature adjustment device 700. The layer plate 200 is provided with a heat preservation area 211 for containing gas, and is provided at different heights of the frame body 600 to form a multi-layer space. The breeding box 100 is suspended on the layer plate 200 and is located between an upper one of the layer plate 200 and a lower one of the layer plate 200 for breeding reptiles. The heating element 300 is provided between the breeding box 100 and the layer plate 200 for heating a bottom of the breeding box 100 above the heating element 300 and for covering the heat preservation area 211. The temperature adjustment device 700 and the heating element 300 are connected to each other wirelessly or wiredly, and is used to adjust a temperature of the heating element 300.

The structure and mutual relationship of the layer plate 200, the breeding box 100 and the heating element 300 can refer to the description of the above-mentioned reptile pet breeding cabinet assembly. In addition, in the present embodiment, multiple layer plates 200 are provided side by side at the same height and one heating element 300 is provided on the multiple layer plates 200 on the same layer. Each of the multiple layer plates 200 corresponds to a breeding box 100.

In order to facilitate a movement of the reptile pet breeding cabinet, a plurality of wheels 800 are provided on a bottom side of the frame body 600, and specifically, the plurality of wheels 800 are universal self-locking wheels.

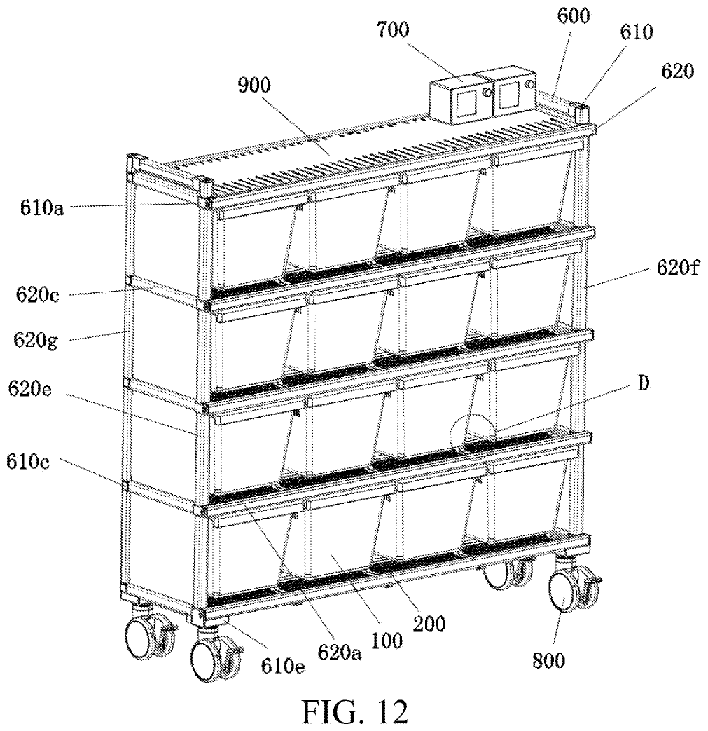

Referring to FIGS. 11 and 12, the frame body 600 includes a plurality of left front connecting members 610a, a plurality of right front connecting members 610b, a plurality of left rear connecting members 610c, a plurality of right rear connecting members 610d, a plurality of front horizontal connecting rods 620a, a plurality of rear horizontal connecting rods 620b, a plurality of left horizontal connecting rods 620c, a plurality of right horizontal connecting rods 620d, a plurality of left front vertical connecting rods 620e, a plurality of right front vertical connecting rods 620f, a plurality of left rear vertical connecting rods 620g, and a plurality of right rear vertical connecting rods 620h. After these components are disassembled, it is convenient for packaging and transportation. When the user wants to use it, the plurality of connecting rods 620 are inserted into the corresponding connecting members 610 to form the frame body 600, and assembly is also more convenient.

The left front connecting member 610a has inserting blocks facing a right side, a rear side, a upper side and a lower side of the reptile pet breeding cabinet, respectively. The right front connecting member 610b has the inserting blocks facing the left side, the rear side, the upper side and the lower side of the reptile pet breeding cabinet, respectively. The left rear connecting member 610c has inserting blocks facing the right side, the front side, the upper side and the lower side of the reptile pet breeding cabinet, respectively. The right rear connecting member 610d has inserting blocks facing the left side, the front side, the upper side and the lower side of the reptile pet breeding cabinet, respectively. One end of the front horizontal connecting rod 620a is sleeved outside the left front connecting member 610a, and the other end of the front horizontal connecting rod 620a is sleeved on the right front connecting member 610b on the same layer. One end of the rear horizontal connecting rod 620b is sleeved outside the left rear connecting member 610c, and the other end of the rear horizontal connecting rod 620b is sleeved on the right front connecting member 610b on the same layer. One end of the left horizontal connecting rod 620c is sleeved outside the left front connecting member 610a, and the other end of the left horizontal connecting rod 620c is sleeved on the left rear connecting member 610c on the same layer. One end of the right horizontal connecting rod 620d is sleeved outside the right front connecting member 610b, and other end is of the right horizontal connecting rod 620d sleeved on the right rear connecting member 610d on the same layer. One end of the left front vertical connecting rod 620e is sleeved outside the left front connecting member 610a, and the other end of the left front vertical connecting rod 620e is sleeved outside the other adjacent left front connecting member 610a. One end of the right front vertical connecting rod 620f is sleeved outside the right front connecting member 610b, and the other end of the right front vertical connecting rod 620f is sleeved outside the other adjacent right front connecting member 610b. One end of the left rear vertical connecting rod 620g is sleeved outside the left rear connecting member 610c, and the other end of the left rear vertical connecting rod 620g is sleeved outside the other adjacent the left rear connecting member 610c. One end of the right rear vertical connecting rod 620h is sleeved outside the right rear connecting member 610d, and the other end of the right rear vertical connecting rod 620h is sleeved outside the other adjacent right rear connecting member 610d.

In the present embodiment, a four-layered frame body 600 with four breeding boxes 100 in each frame body 600 is used as an example for explanation. This reptile pet breeding cabinet has sixteen breeding boxes 100, twenty layer plates 200 and four heating elements 300. An upper side of the uppermost layer plate 200 is no longer provided with the heating element 300, and only one pressing plate 900 that can seal the heat preservation area 211 of the layer plate 200 is provided.

The frame body 600 includes five left front connecting members 610a, five right front connecting members 610b, five left rear connecting members 610c, five right rear connecting members 610d, five front horizontal connecting rods 620a, five rear horizontal connecting rods 620b, five left horizontal connecting rods 620c, five right horizontal connecting rods 620d, four left front vertical connecting rods 620e, four right front vertical connecting rods 620f, four left rear vertical connecting rods 620g and four right rear vertical connecting rods 620h. The uppermost one and the lowermost one of the connecting members 610 may be provided differently. For example, the uppermost one of the connecting members 610 may not be provided with the inserting block facing the upper side, and the lowermost one of the connecting members 610 is not provided with the inserting block facing the lower side.

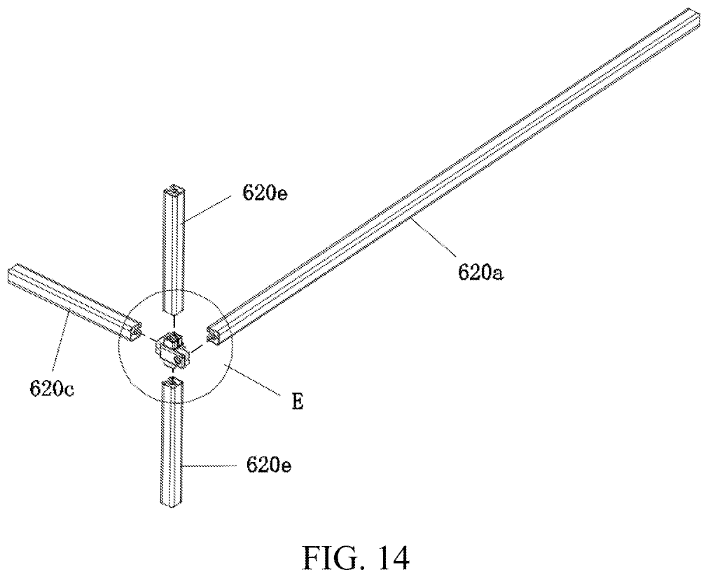

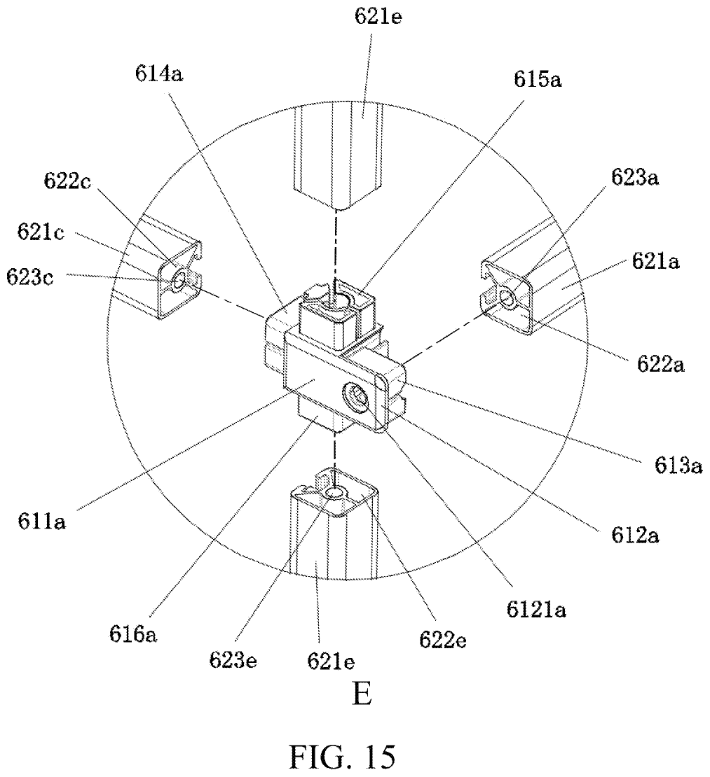

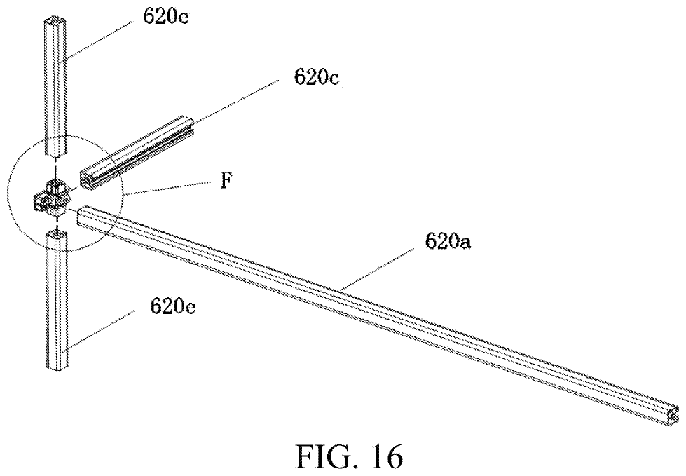

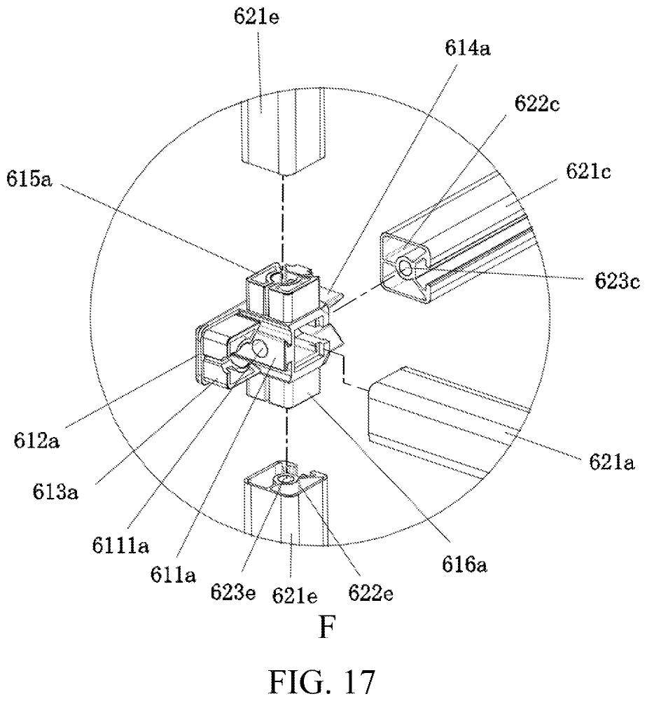

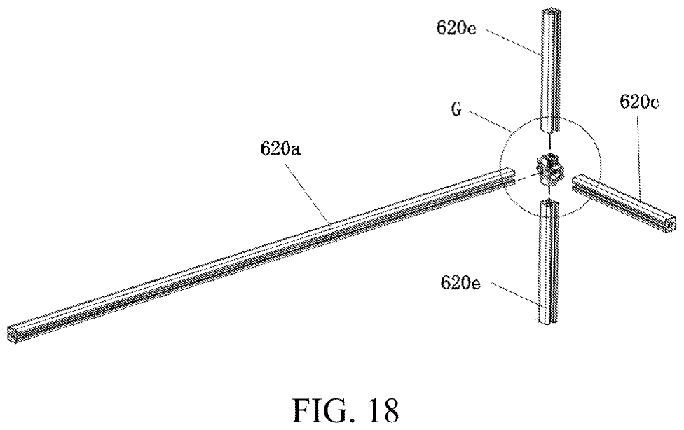

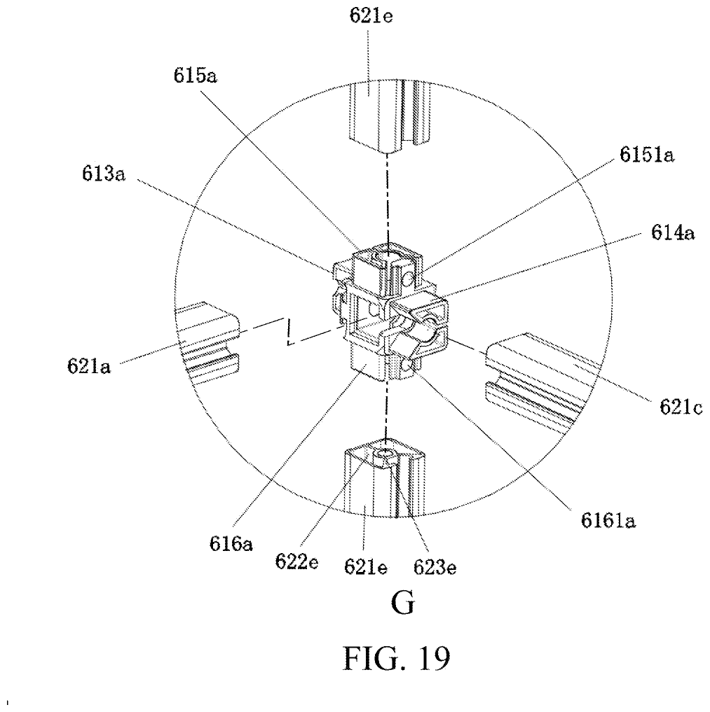

Referring to FIGS. 14 to 19, specifically, in the present embodiment, the left front connecting member 610a includes a base body 611a that is cube-shaped, an extension plate 612a, a right inserting block 613a, a rear inserting block 614a, an upper inserting block 615a, and a lower inserting block 616a. The extension plate 612a extends forward from a left end surface of the base body 611a, and the extension plate 612a is aligned with an end of the front horizontal connecting rod 620a. The right inserting block 613a protrudes to the right side from a right end surface of the extension plate 612a for providing the front horizontal connecting rods 620a to be inserted into. The rear inserting block 614a protrudes rearward from a rear end surface of the base body 611a for providing the left horizontal connecting rod 620c to be inserted into. The upper inserting block 615a protrudes upward from an upper end surface of the base body 611a for providing the left front vertical connecting rod 620c to be inserted into. The upper inserting block 615a protrudes upward from the upper end surface of the base body 611a for providing the left front vertical connecting rod 620e to be inserted into. The lower inserting block 616a protrudes downward from a lower end surface of the base body 611a for providing the left front vertical connecting rod 620e to be inserted into.

The structure of the right front connecting member 610b is similar to those described above, except that the right inserting block is replaced with a left inserting block. That is, the right front connecting member 610b includes the base body that is cube-shaped, the extension plate, the left inserting block, the rear inserting block, the upper inserting block, and the lower inserting block. The extension plate extends forward from the right end of the base body, and the extension plate is aligned with the end of the front horizontal connecting rod 620a. The left inserting block protrudes to the left side from the left end surface of the extension plate for providing the front horizontal connecting rod 620a to be inserted into. The rear inserting block protrudes backward from the rear end of the base body for providing the left horizontal connecting rod 620c to be inserted into. The upper inserting block protrudes upward from the upper end of the base body for providing the left front vertical connecting rod 620e to be inserted into. The lower inserting block protrudes downward from the lower end of the base body for providing the left front vertical connecting rod 620e to be inserted into.

Referring to FIGS. 11 to 19, the front horizontal connecting rod 620a, the rear horizontal connecting rod 620b, the left horizontal connecting rod 620c, the right horizontal connecting rod 620d, the left front vertical connecting rod 620e, the right front vertical connecting rod 620f, the left rear vertical connecting rod 620g and the right rear vertical connecting rod 620h are all cuboid-shaped and have a square cross-section.

Since the base body 611a of the left front connecting member 610a (the same as the right front connecting member 610b) is in the shape of a cube, and the extension plate 612a extends forward from the left end face or the right end face, so that after the left front connecting member 610a and the right front connecting member 610b are inserted and assembled, when viewed from the front, only the front horizontal connecting rod 620a can be seen, and the left front connecting member 610a and the right front connecting member 610b cannot be seen, thereby making the front side of the frame body 600 more integrated and more beautiful.

Referring to FIGS. 14 to 19, the front horizontal connecting rod 620a, the left horizontal connecting rod 620c and the left front vertical connecting rod 620e are taken as an example. The front horizontal connecting rod 620a includes a rod body 621a. The rod body 621a is provided with a plurality of through-slots 622a in a circle along a length direction of the rod body 621a for insertion. One of the plurality of through-slots 622a is not closed in the length direction of the rod body 621a and a locking hole 623a is provided between the plurality of through-slots 622a. The left horizontal connecting rod 620c includes a rod body 621c. The rod body 621c is provided with a plurality of through-slots 622c in a circle along a length direction of the rod body 621c for insertion. One of the plurality of through-slots 622c is not closed in the length direction of the rod body 621c, and a locking hole 623c is provided between the plurality of through-slots 622c. The left front vertical connecting rod 620e includes a rod body 621e. The rod body 621e is provided with a plurality of through-slots 622e in a circle along a length direction of the rod body 621e for insertion. One of the plurality of through-slots 622e is not closed in the length direction of the rod body 621e, and a locking hole 623e is provided between the plurality of through-slots 622e.

Referring also to FIGS. 14 to 19, the left front connecting member 610a is provided with two right inserting blocks 613a, and the extension plate 612a is provided with a first through hole 6121a between the two right inserting blocks 613a. When the front horizontal connecting rod 620a is inserted into the left front connecting member 610a, a locking member is used to pass through the first through hole 6121a and the locking hole 623a of the front horizontal connecting rod 620a for locking. The left front connecting member 610a is provided with two rear inserting blocks 614a. The base body 611a is provided with a second through hole 6111a between the two rear inserting blocks 614a. When the left horizontal connecting rod 620c is inserted into the left front connecting member 610a, the locking member is used to pass through the second through hole 6111a and the locking hole 623a of the left horizontal connecting rod 620c for locking. The left front connecting member 610a is provided with three upper inserting blocks 615a that form a circle. The lower inserting block 616a corresponding to the through-slot 622e that is not closed in the length direction is provided with a fourth through hole 6161a in a horizontal direction. When an upper end of the left front vertical connecting rod 620e is inserted into the left front connecting member 610a, the locking member is used to pass through the fourth through hole 6161a for locking.

As can be seen from the above, four through holes are provided on the left front connecting member 610a, and the four through holes are respectively connected to the locking hole 623a, the locking hole 623c and the locking holes 623e respectively provided on the front horizontal connecting rod 620a, the left horizontal connecting rod 620c and the two left front vertical connecting rods 620e, and then the locking holes are combined with the connecting rods 620 through the locking members. In the present embodiment, the locking member is preferably a jackscrew (not shown in the figures), and in other embodiments, other locking members may also be used.

The first through hole 6121a corresponds to a left end surface of the front horizontal connecting rod 620a. The first through hole 6121a is provided on the extension plate 612a. The user only needs to drive the locking member from the left end to the right of the extension plate 612a. The second through hole 6111a corresponds to a front end surface of the left horizontal connecting rod 620c. The second through hole 6111a is provided in the base body 611a.

The user only needs to drive the locking member backward from a front end of the base body 611a. The third through hole 6151a corresponds to a lower end surface of the front left vertical connecting rod 620e. The third through hole 6151a is provided in the upper inserting block 615a. The user only needs to drive the locking member forward from a rear end of the upper inserting block 615a. The third through hole 6151a corresponds to an upper end surface of the left front vertical connecting rod 620e. The fourth through hole 6161a is provided in the lower inserting block 616a. The user only needs to drive the locking member forward from a rear end of the lower inserting block 616a.

The position design of each through hole makes it easy for the user to insert the locking members, and except for the first through hole 6121a, the other through holes are hidden inside after the entire reptile pet breeding cabinet is assembled, so that the beauty of the pet breeding cabinet is not affected.

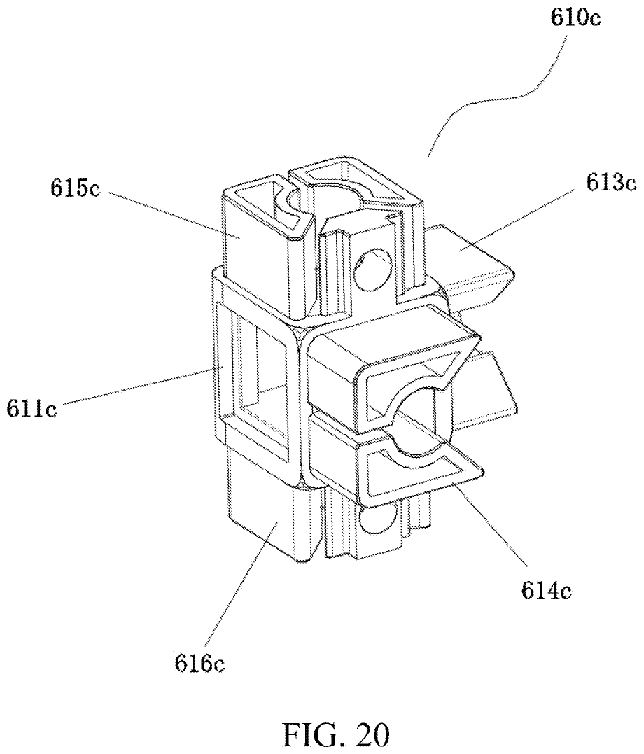

Referring to FIG. 20, in this embodiment, the left rear connecting member 610c includes a base body 611c that is cube-shaped, a right inserting block 613c, a front inserting block 614c, an upper inserting block 615c and a lower inserting block 616c. The right inserting block 613c protrudes from a right end surface of the base body 611c to the right. The front inserting block 614c protrudes forward from a front end surface of the base body 611c. The upper inserting block 615c protrudes upward from an upper end surface of the base body 611c. The lower inserting block 616c protrudes downward from a lower end surface of the base body 611c. Since the rear side of the frame body 600 is adjacent to the wall, a simpler left rear connecting member 610c can be used without overly considering the appearance. Certainly, in certain embodiments, the left rear connecting member 610c and the right rear connecting member 610d can also be configured to have a structure similar to the left front connecting member 610a and the right front connecting member 610b.

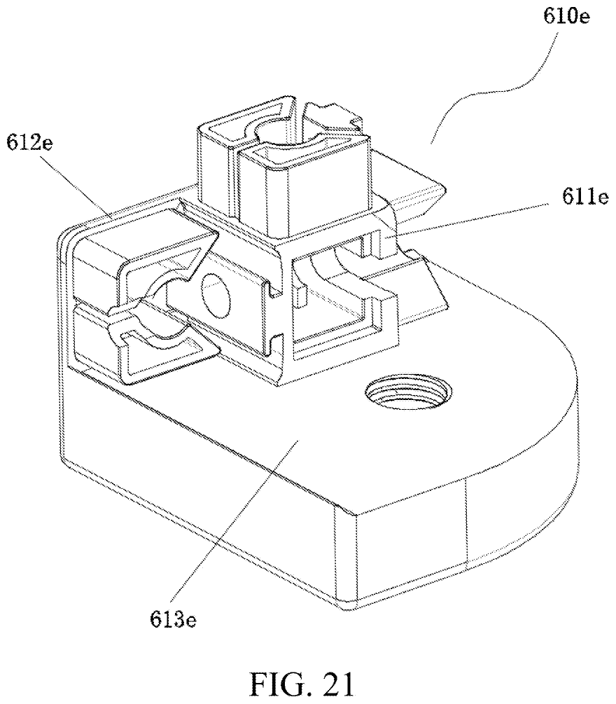

Referring to FIG. 21, in the present embodiment, the lowermost left front connecting member 610a (i.e., the lower left front connecting member 610e) is no longer provided with the lower inserting block, and is provided with wheel connecting plate 613e extending to the right at lower ends of the base body 611e and the extension plate 612e. Threaded holes are provided on the wheel connecting plate 613e, and the threaded holes are used for threading connection with the studs on the wheels 800 to fix the wheels 800 below the wheel connecting plate 613e.

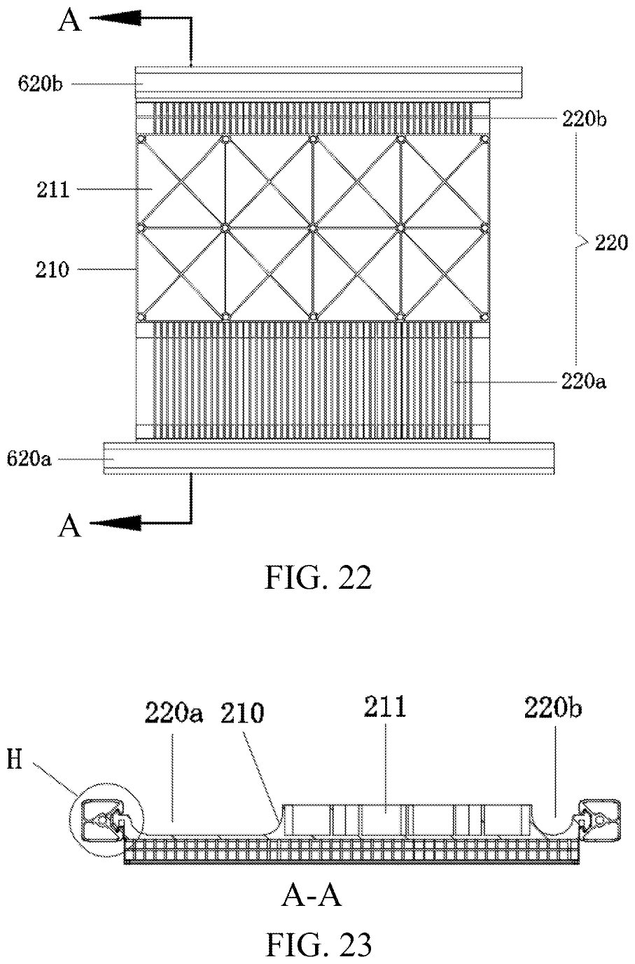

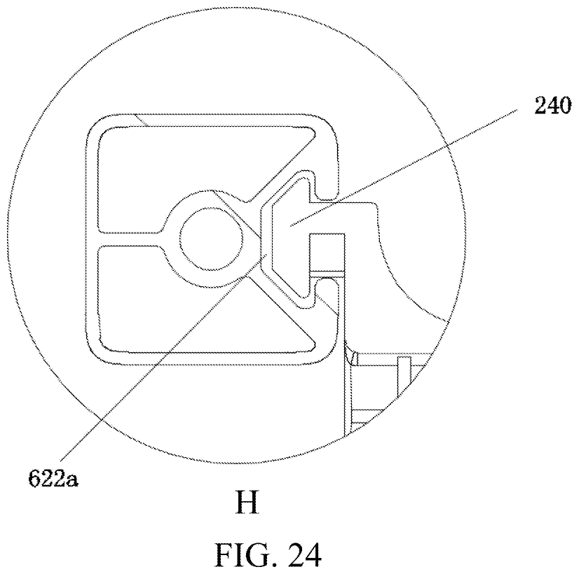

Referring to FIGS. 22 to 24, each of a front side and a rear side of the layer plate 200 is provided with a sliding bar 240 corresponding to the through-slot 622a that is not closed in the length direction. When the layer plate 200 is slidably assembled between the front horizontal connecting rod 620a and the rear horizontal connecting rod 620b, the layer plate 200 is suspended on the frame body 600 by the front horizontal connecting rod 620a and the rear horizontal connecting rod 620b. A blocking structure is provided between the through-slot 622a and the sliding bar 240, so that the layer plate 200 can only slide into the space between the front horizontal connecting rod 620a and the rear horizontal connecting rod 620b from the left and right directions. The reptile pet breeding cabinet is assembled as follows.

Step 1: slidably assembling the plurality of layer plates 200 between the front horizontal connecting rod 620a and the rear horizontal connecting rod 620b;

Step 2: assembling the left front connecting member 610a and the left rear connecting member 610c respectively at two ends of the left horizontal connecting rod 620c, and in addition, assembling the right front connecting member 610b and the right rear connecting member 610d respectively at two ends of the right horizontal connecting rod 620d, in which the order of Step 1 and Step 2 can be reversed, or Step 1 and Step 2 can be performed simultaneously;

Step 3: assembling the layer plate 200, the front horizontal connecting rod 620a and the rear horizontal connecting rod 620b assembled in Step 1, the left horizontal connecting rod 620c, the left front connecting member 610a and the left rear connecting member 610c assembled in Step 2, and the right horizontal connecting rod 620d, the right front connecting member 610b and the right rear connecting member 610d to obtain a complete layer;

Step 4: repeating Steps 1 to 3;

Step 5: using the left front vertical connecting rod 620e, the right front vertical connecting rod 620f, the left rear vertical connecting rod 620g and the right rear vertical connecting rod 620h to connect and assemble the different layer plates; and

Step 6: suspending the breeding box 100 and placing the heating element 300.

Referring to FIGS. 11, 25, and 26, a temperature sensor 320 is provided on the heating element 300, and the temperature adjustment device 700 is provided with a display screen. The temperature adjustment device 700 is used to adjust a temperature and then display the temperature on the display screen. The temperature adjustment device 700 and the plurality of heating elements 300 are independently controlled. In other embodiments, the temperature sensor 320 may also be provided on the breeding box 100.

In the present disclosure, a thyristor can also be used to achieve precise temperature control, and a relay can be connected in series to achieve over-temperature protection. When the temperature sensor 320 detects that the temperature is higher than a predetermined temperature and the heating continues, the heating power will be cut off by the relay.

When the temperature returns below the predetermined temperature, the temperature is controlled by the switching time of the relay. Even after the thyristor fails, the temperature can be maintained stable and the life safety of the reptile can be protected.

In addition, the reptile pet breeding cabinet is provided with a light sensor and/or a time module for signal connection with the temperature adjustment device 700. The temperature adjustment device 700 adjusts the temperature of the heating element 300 through the signals fed back by the light sensor and/or time module, thereby adjusting the temperature inside the breeding box 100. Nighttime cooling can be set based on time and/or light to determine if it is nighttime. The temperature difference between day and night is set closer to the temperature of the original place of production, which can improve the physical fitness of the reptile.

The above is only used to illustrate the technical solution of the present disclosure without limiting it. Other modifications or equivalent substitutions made by those of ordinary skill in the art to the technical solution of the present disclosure shall be covered by the claims of the present disclosure as long as they do not depart from the spirit and scope of the technical solution of the present disclosure.

What is claimed is:

1. A reptile pet breeding cabinet assembly, comprising:
a breeding box (100) configured to breed reptiles;
a layer plate (200) provided on a lower side of the breeding box (100), wherein a heat preservation area (211) for containing gas is provided on the layer plate (200); and
a heating element (300) provided between the breeding box (100) and the layer plate (200), and configured to heat a bottom of the breeding box (100) and to cover the heat preservation area (211).

2. The reptile pet breeding cabinet assembly according to claim 1, wherein the layer plate (200) includes a bottom layer plate (200a) and a top layer plate (200b), and wherein the bottom layer plate (200a) is provided on the lower side of the breeding box (100), the top layer plate (200b) is provided on an upper side of the breeding box (100) and is configured to suspend the breeding box (100), and the top layer plate (200b) is configured to be the bottom layer (200a) of another breeding box (100) arranged above the breeding box (100).

3. The reptile pet breeding cabinet assembly according to claim 2, wherein a convex structure (210) is provided on the layer plate (200), the heat preservation area (211) is formed in the convex structure (210), the layer plate (200) is provided with a ventilation area (220) on a side of the heat preservation area (211), and a top side of the convex structure (210) is higher than the ventilation area (220).

4. The reptile pet breeding cabinet assembly according to claim 3, wherein the ventilation area (220) includes a front ventilation area (220a) located on a front side of the heat preservation area (211), and a rear ventilation area (220b) located on a rear side of the heat preservation area (211).

5. The reptile pet breeding cabinet assembly according to claim 4, wherein the front ventilation area (220a) is configured to be adjacent to a pedestrian passage, and the heating element (300) is a flexible heating sheet located on a side away from the pedestrian passage.

6. The reptile pet breeding cabinet assembly according to claim 3, wherein a side of the ventilation area (220) connected to the convex structure (210) is in an arc-shaped transition.

7. The reptile pet breeding cabinet assembly according to claim 2, wherein a sliding slot (230) is provided on each of a left side and a right side of a lower part of the layer plate (200), an outer edge (110) slidably assembled on the sliding slot (230) is provided one each of a left side and a right side of an upper part the breeding box (100), and the breeding box (100) is configured to be suspended on the top layer plate (200b) and capable of being pulled outward relative to the top layer plate (200b).

8. The reptile pet breeding cabinet assembly according to claim 2, further comprising: a box cover (400) covering the upper side of the breeding box (100) and located on a lower side of the top layer plate (200b), wherein a plurality of ventilation holes are provided on the box cover (400).

9. The reptile pet breeding cabinet assembly according to claim 8, wherein an overlapping rib (130) that protrudes inward is provided on an inner wall of the breeding box (100) adjacent to the upper side of the breeding box (100), and the box cover (400) is configured to be placed on the overlapping rib (130).

10. The reptile pet breeding cabinet assembly according to claim 9, wherein a limiting notch (131) is provided on the overlapping rib (130), and a limiting protrusion (410) corresponding to the limiting notch (131) is provided on a bottom side of the box cover (400).

11. The reptile pet breeding cabinet assembly according to claim 1, further comprising: a partition (500) configured to divide the breeding box (100) into at least two spaces.

12. A reptile pet breeding cabinet, comprising:
a frame (600);
a plurality of layer plates (200) provided at different heights of the frame (600) to form a multi-layer space, wherein heat preservation areas (211) for containing gas are respectively provided on the plurality of layer plates (200);
a plurality of breeding boxes (100) correspondingly suspended on the plurality of layer plates (200), wherein each of the plurality of breeding boxes (100) is located between an upper one of the layer plate (200) and a lower one of the layer plate (200) and configured to breed reptiles;
a plurality of heating elements (300) correspondingly provided between the plurality of breeding boxes (100) and the plurality of layer plates (200), and configured to heat bottoms of the plurality of breeding boxes (100) respectively arranged above the plurality of heating elements (300) and configured to cover the heat preservation areas (211); and
a temperature adjustment device (700) correspondingly and wirelessly or wiredly connected to the plurality of heating elements (300), and configured to adjust temperatures of the plurality of heating elements (300).

13. The reptile pet breeding cabinet according to claim 12, wherein multiple layer plates (200) are provided side by side at a same height, one of the plurality of heating elements (300) is provided on one of the multiple layer plates (200) on a same layer, and each of the multiple heating elements (300) corresponds to a corresponding one of the plurality of breeding boxes (100).

14. The reptile pet breeding cabinet according to claim 12, wherein the frame (600) includes:
a plurality of left front connecting members (610a) each having inserting blocks respectively facing a right side, a rear side, an upper side and a lower side of the reptile pet breeding cabinet;
a plurality of right front connecting members (610b) each having the inserting blocks respectively facing the left side, the rear side, the upper side and the lower side of the reptile pet breeding cabinet;
a plurality of left rear connecting members (610c) each having the inserting blocks respectively facing the right side, the front side, the upper side and the lower side of the reptile pet breeding cabinet;
a plurality of right rear connecting members (610d) each having the inserting blocks respectively facing the left side, the front side, the upper side and the lower side of the reptile pet breeding cabinet;
a plurality of front horizontal connecting rods (620a) each having one end sleeved outside the left front connecting member (610a), and another end sleeved on the right front connecting member (610b) on a same layer;
a plurality of rear horizontal connecting rods (620b) each having one end sleeved outside the left rear connecting member (610c), and another end sleeved on the right rear connecting member (610d) on a same layer;
a plurality of left horizontal connecting rods (620c) each having one end sleeved outside the left front connecting member (610a), and another end sleeved on the left rear connecting member (610c) on a same layer;
a plurality of right horizontal connecting rods (620d) each having one end sleeved outside the right front connecting member (610b), and another end sleeved on the right rear connecting member (610d) on a same layer;
a plurality of left front vertical connecting rods (620e) each having one end sleeved outside one of the plurality of left front connecting members (610a), and another end sleeved outside another of the plurality of left front connecting members (610a) that is adjacent to the left front vertical connecting rod (620e);
a plurality of right front vertical connecting rods (620f) each having one end sleeved outside one of the plurality of right front connecting members (610b), and another end sleeved outside another of the plurality of right front connecting members (610b) that is adjacent to the right front vertical connecting rod (620f);
a plurality of left rear vertical connecting rods (620g) each having one end sleeved outside one of the plurality of left rear connecting members (610c), and another end sleeved outside another of the plurality of left rear connecting members (610c) that is adjacent to the left rear vertical connecting rod (620g); and
a plurality of right rear vertical connecting rods (620h) each having one end sleeved outside one of the plurality of right rear connecting members (610d), and another end sleeved outside another of the plurality of right rear connecting members (610d) that is adjacent to the right rear vertical connecting rod (620h).

15. The reptile pet breeding cabinet according to claim 14, wherein each of the plurality of left front connecting members (610a) includes:
a base body (611a) in the shape of a cube;
an extension plate (612a) extending forward from a left end surface of the base body (611a), wherein the extension plate (612a) is aligned with an end of one of the plurality of front horizontal connecting rods (620a);
a right inserting block (613a) protruding from a right end surface toward a right side of the extension plate (612a) and configured for the front horizontal connecting rod (620a) to be inserted into; a rear inserting block (614a) protruding rearward from a rear end surface of the base body (611a) and configured for the left horizontal connecting rod (620c) to be inserted into;
an upper inserting block (615a) protruding upward from an upper end surface of the base body (611a) and configured for the left front vertical connecting rod (620e) to be inserted into; and
a lower inserting block (616a) protruding downward from a lower end surface of the base body (611a) and configured for the left front vertical connecting rod (620e) to be inserted into;
wherein the plurality of front horizontal connecting rods (620a), the plurality of rear horizontal connecting rods (620b), the plurality of left horizontal connecting rods (620c), the plurality of right horizontal connecting rods (620d), the plurality of left front vertical connecting rods (620e), the plurality of right front vertical connecting rods (620f), the plurality of left rear vertical connecting rods (620g) and the plurality of right rear vertical connecting rods (620h) are all cuboid-shaped and have a square cross-section.

16. The reptile pet breeding cabinet according to claim 15, wherein the plurality of front horizontal connecting rods (620a), the plurality of rear horizontal connecting rods (620b), the plurality of left horizontal connecting rods (620c), the plurality of right horizontal connecting rods (620d), the plurality of left front vertical connecting rods (620e), the plurality of right front vertical connecting rods (620f), the plurality of left rear vertical connecting rods (620g) and the plurality of right rear vertical connecting rods (620h) all include a rod body (621a, 621c, 621e), wherein a plurality of through-slots (622a, 622c, 622e) formed in a circle along a length direction and configured for inserting are provided on the rod body (621a, 621c, 621e), and one of the plurality of through-slots (622a, 622c, 622e) is not closed in the length direction, and a locking hole (623a, 623c, 623e) is provided between the plurality of through-slots (622a, 622c, 622e).

17. The reptile pet breeding cabinet according to claim 16, wherein two right inserting blocks (613a) are provided, and a first through hole (6121a) between the two right inserting blocks (613a) is provided on the extension plate (612a), and, when the front horizontal connecting rod (620a) is inserted into the left front connecting member (610a), a locking member is used to pass through the first through hole (6121a) and the locking hole (623a) of the front horizontal connecting rod (620a) for locking;
wherein two rear inserting blocks (614a) are provided, a second through hole (6111a) between the two rear inserting blocks (614a) is provided on the base body (611a), and, when the left horizontal connecting rod (620c) is inserted into the left front connecting member (610a), the locking member is used to pass through the second through hole (6111a) and the locking hole (623c) of the left horizontal connecting rod (620c) for locking;
wherein three upper inserting blocks (615a) are provided in a circle, and a third through hole (6151a) is provided on one of the upper inserting blocks (615a) corresponding to the through-slot (622e) that is not closed in the length direction in a horizontal direction, and, when a lower end of the left front vertical connecting rod (620e) is inserted into the left front connecting member (610a), the locking member is used to pass through the third through hole (6151a) for locking; and
wherein three lower inserting blocks (616a) are provided in a circle, and a fourth through hole (6161a) is provided on one of the lower inserting block (616a) corresponding to the through-slot (622e) that is not closed in the length direction in the horizontal direction, and, when an upper end of the left front vertical connecting rod (620e) is inserted into the left front connecting member (610a), the locking member is used to pass through the fourth through hole (6161a) for locking.

18. The reptile pet breeding cabinet according to claim 16, wherein a sliding bar (240) is provided on each of a front side and a rear side of the layer plate (200) corresponding to the through-slot (622a) that is not closed in the length direction, and, when the layer plate (200) is slidably assembled between the front horizontal connecting rod (620a) and the rear horizontal connecting rod (620b), the layer plate (200) is suspended on the frame body (600) by the front horizontal connecting rod (620a) and the rear horizontal connecting rod (620b).

19. The reptile pet breeding cabinet according to claim 12, wherein a temperature sensor (320) is provided on each of the plurality of heating elements (300) and/or the breeding box (100), a display screen is provided on the temperature adjustment device (700), the temperature adjustment device (700) is configured to adjust a temperature and display the temperature on the display screen, and the temperature adjustment device (700) and the plurality of heating elements (300) are independently controlled.

20. The reptile pet breeding cabinet according to claim 19, wherein a light sensor and/or a time module signally connected to the temperature adjustment device (700) is provided on the reptile pet breeding cabinet, the temperature adjustment device (700) adjusts temperatures of the plurality of heating elements (300) through signals fed back by the light sensor and/or the time module, so that temperatures in the plurality of breeding boxes (100) are adjusted.
